(19) United States
(12) Patent Application Publication
(10) Pub. No.: US 2006/0153900 A1
(43) Pub. Date: Jul. 13, 2006

(54) Dietary supplement for treatment of lipid risk factors

(76) Inventor: Joel J. Pins, Minneapolis, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/328,658

(22) Filed: Jan. 10, 2006

Related U.S. Application Data
(60) Provisional application No. 60/643,487, filed on Jan. 13, 2005.

(51) Int. Cl. A61K 31/455; A61K 9/68; A61K 9/48; A61K 9/20

(52) U.S. Cl. 424/440; 424/451; 424/464; 514/356

(57) ABSTRACT

A dietary supplement is provided for the treatment of lipid risk factors comprising nicotinic acid, pantethine and lemon/orange flavonates. This supplement provides significant benefit in affect on major cardiovascular disease (“CVD”) lipid risk factors, including LDL-C, HDL-C, and Tg in a non-prescription formulation. Also described are methods for treatment of lipid risk factors in a person in need thereof, comprising administering a dietary supplement in unit dosage form comprising nicotinic acid, pantethine and lemon/orange flavonates to the person.

This application claims the benefit of U.S. Provisional Application Ser. No. 60/643,487, filed Jan. 13, 2005, entitled “DIETARY SUPPLEMENT FOR TREATMENT OF LIPID RISK FACTORS,” which application is incorporated herein by reference in its entirety.

### BACKGROUND OF THE INVENTION

The Centers for Disease Control reports that heart disease and stroke—the principal components of cardiovascular disease—are the first and third leading causes of death for both men and women in the United States, accounting for nearly 40% of all deaths. Over 930,000 Americans die of cardiovascular disease each year, which amounts to one death every 34 seconds. In addition, the CDC reported that the cost of heart disease and stroke in the United States was projected to be $368 billion in 2004, including health care expenditures and lost productivity from death and disability.

It is recognized that one can is to reduce the risk of cardiovascular disease through therapy aimed at lowering the blood lipid levels. Various therapies, including the use of pharmaceutical formulations, are currently extensively used by patients in need. There is a desire to find non-pharmaceutical (e.g. “over the counter”) formulations and supplements to provide assist in the management of lipid risk factors. For example, U.S. Pat. No. 6,632,428 to Zhang, et al. discloses methods and compositions comprising red rice fermentation products that can be used as natural dietary supplements and/or medicaments for the treatment or prevention of hyperlipidemia and associated disorders and symptoms, such as cardiovascular diseases.

### SUMMARY OF THE INVENTION

The present invention provides a dietary supplement for the treatment of lipid risk factors comprising nicotinic acid, pantethine and lemon/orange flavonates. This supplement provides significant benefit in affect on major cardiovascular disease (“CVD”) lipid risk factors, including LDL-C, HDL-C, and Tg. Also described are methods for treatment of lipid risk factors in a person in need thereof, comprising administering a dietary supplement in unit dosage form comprising nicotinic acid, pantethine and lemon/orange flavonates to the person.

It is believed that provision of the three listed active ingredients in a single unit dose formulation provides substantial benefit in co-action of the active ingredients, as well as convenience to the user. It is particularly advantageous that CVD factor benefits are provided by the present dietary supplement in a non-prescription formulation.

### DETAILED DESCRIPTION

The essential ingredients of the dietary supplement of the present invention comprise nicotinic acid, pantethine and lemon/orange flavonates. All individual ingredients have been clinically tested in randomized clinical trials and have been shown to beneficially and safely improve CVD risk. Nicotinic acid and pantethine have also been shown to improve non-traditional CVD risk factors such as apolipoprotein B, LDL particle size, and Lp(a)-C levels. Lemon/orange flavonates are an excellent source of polymethoxylated flavones (PMFs). Animal and human studies have shown that PMFs lower LDL-C and also likely possess other CVD benefits, such as improved platelet and vascular function.

It will be appreciated that the actual preferred amounts of active compound in a specific case will vary according to the particular compositions formulated, the mode of application, and the nature of the person being treated. For example, the specific dose for a particular patient depends on the age, body weight, general state of health, on the diet, on the timing and mode of administration, on the rate of excretion, and on medicaments used by the patient.

In a preferred embodiment, the dietary supplement comprises
a) from about 1 to about 1000 mg Nicotinic Acid,
b) from about 1 to about 1000 mg Pantethine, and
c) from about 1 to about 1000 mg lemon/orange flavonates.

In another preferred embodiment, the dietary supplement comprises
a) from about 50 to about 500 mg Nicotinic Acid,
b) from about 50 to about 400 mg Pantethine, and
c) from about 25 to about 350 mg lemon/orange flavonates.

In yet another preferred embodiment, the dietary supplement comprises
a) from about 100 to about 350 mg Nicotinic Acid,
b) from about 100 to about 300 mg Pantethine, and
c) from about 50 to about 250 mg lemon/orange flavonates.

As a preferred example of a dietary supplement, a tablet is provided that comprises:
a) about 250 mg Nicotinic Acid,
b) about 200 mg Pantethine, and
c) about 150 mg lemon/orange flavonates.

The active ingredients as discussed above can optionally be combined with any additional ingredients that do not adversely affect the treatment of lipid risk factors function of the dietary supplement. In a preferred embodiment, the dietary supplement “consists of” the above active ingredients, or in other words does not contain active ingredients other than the above recited three ingredients. A three-active-component formulation is advantageous in providing a simple formula with a minimum potential of adverse interactions with other materials.

The dietary supplement of the present invention preferably is formulated with one or more nontoxic pharmaceutically acceptable carriers, such as cornstarch, lactose, or sucrose, which do not deleteriously react with the active compounds.

Preferably, the dietary supplement of the present invention is provided in a format suitable for oral administration, and more preferably in a dry oral administration format. Most preferably, the dietary supplement is provided in tablet form. In an alternative preferred format, the dietary supplement is in a form selected from the group consisting of dragees, lozenges, powders, or capsules.

In a particularly preferred embodiment, the dietary supplement of the present invention is provided in a sustained release or time release form. For purposes of the present invention the dietary supplement is considered to be “sustained release” or “time release” if the active ingredients are delivered to the bloodstream at a rate that is measurably longer than the rate of a like conventional supplement administration form. Preferably, the active ingredients are delivered to the bloodstream at a rate that is at least about twice as long as the rate of delivery of a like conventional supplement administration form.

The provision of a dietary supplement in sustained release form finds particular benefit as a convenient dosage form by virtue of eliminating the necessity for dosage several times during the day. Moreover, therapeutic benefits may also be obtained by the sustained release of the active ingredients of the inventive formulation. In one aspect, the sustained-release form beneficially delivers the active agents systemically more slowly, which can improve product tolerability and efficacy. Additionally, it is believed that the sustained release dosage form of the present invention provides superior benefit to like dietary supplements that are not in sustained release form due to the continuous application of the effect of the active ingredient without disadvantageous periods absent the effect of the active ingredient. It is believed that even short time periods absent the effect of the active ingredient have a disproportionately adverse effect in the treatment of lipid risk factors.

The dietary supplement of the present invention may be provided as a sustained release product in any appropriate manner, such as those known in the pharmaceutical and health product arts.

According to one preferred embodiment, a matrix such as wax, hydroxypropyl methylcellulose, or the like, is used as a pharmaceutical adjunct, which provides a sustained release of the active constituents of the tablet. According to this preferred embodiment, a sufficient amount of the time release matrix material can be incorporated in the tablet to ensure proper time release tablet.

A preferred example of a sustained release systems include a proprietary process developed by Innovite, Inc. This process is described at http://www.endur.com/index.cfm?fuseaction=main.about as follows:
Innovite has developed a novel process for impregnating a matrix (vegetable source) with active ingredients. This material is then compressed into tablets having uniform, continuous release rates. This proprietary process uses a cold-extrusion technique that extends stability profiles by eliminating heat, moisture and solvents.

Alternatively, the active ingredients of the dietary supplement can be provided in a suitable discrete from, such as in particle or granule form, and further provided with an enteric coating that is resistant to disintegration in gastric juices.

The coated granules can be mixed with optional additives such as antioxidants, stabilizers, binder, lubricant, processing aids and the like. The mixture can be compacted into a tablet which, prior to use, is hard and dry or it can be poured into a capsule.

Those skilled in the art will also appreciate that the formulations of the present invention may also be encapsulated in other time-release delivery systems such as a liposome delivery system, polysaccharides exhibiting a slow release mechanism, polymer implants or microspheres. In such time release delivery systems, the active compound is suitably protected with differentially degradable coatings, e.g., by microencapsulation, multiple coatings, etc., and such means effect continual dosing of compositions contained therein.

In use, the dietary supplement as described herein is administered to a person in need of treatment of lipid risk factors. In one method, a unit dose dietary supplement is administered on a substantially daily basis. Preferably, the unit dose dietary supplement is in a time release format. In another method, the unit dose dietary supplement is administered in from about two to about six doses per day.

In a preferred embodiment, the dietary supplement of the present invention is administered with food.

All percentages and ratios used herein are weight percentages and ratios unless otherwise indicated. All publications, patents and patent documents cited are fully incorporated by reference herein, as though individually incorporated by reference. Numerous characteristics and advantages of the invention meant to be described by this document have been set forth in the foregoing description. It is to be understood, however, that while particular forms or embodiments of the invention have been illustrated, various modifications can be made without departing from the spirit and scope of the invention.

1. A dietary supplement for treatment of lipid risk factors, comprising a therapeutically effective amount of nicotinic acid, pantethine and lemon/orange flavonates in unit dosage form.

2. The supplement of claim 1, wherein the dietary supplement is in sustained release form.

3. The supplement of claim 1, wherein the dietary supplement is in the form of a dry tablet.

4. The supplement of claim 1, wherein the dietary supplement is in a form selected from the group consisting of dragees, lozenges, powders, or capsules.

5. The supplement of claim 1, wherein the dietary supplement comprises
a) from about 1 to about 1000 mg Nicotinic Acid,
b) from about 1 to about 1000 mg Pantethine, and
c) from about 1 to about 1000 mg lemon/orange flavonates.

6. The supplement of claim 1, wherein the dietary supplement comprises
a) from about 50 to about 500 mg Nicotinic Acid,
b) from about 50 to about 400 mg Pantethine, and
c) from about 25 to about 350 mg lemon/orange flavonates.

7. The supplement of claim 1, wherein the dietary supplement comprises
a) from about 100 to about 350 mg Nicotinic Acid,
b) from about 100 to about 300 mg Pantethine, and
c) from about 50 to about 250 mg lemon/orange flavonates.

8. A method for treatment of lipid risk factors in a person in need thereof, comprising administering a dietary supplement in unit dosage form comprising a therapeutically effective amount of nicotinic acid, pantethine and lemon/orange flavonates to the person.

9. The method of claim 8, where the unit dose dietary supplement is administered on a substantially daily basis.

10. The method of claim 8, where the unit dose dietary supplement is in a time release format.

11. The method of claim 8, where the unit dose dietary supplement is administered in from about two to about six doses per day.
